(19) United States
(12) Patent Application Publication
(10) Pub. No.: US 2005/0043253 A1
(43) Pub. Date: Feb. 24, 2005

(54) Use of wound healing compositions for prevention of infections and allergies

(76) Inventor: Bradley R. Cook, Sharon, MA (US)

Correspondence Address: NUTTER MCCLENNEN & FISH LLP, WORLD TRADE CENTER WEST, 155 SEAPORT BOULEVARD, BOSTON, MA, 02210-2604, US

(21) Appl. No.: 10/920,020

(22) Filed: Aug. 17, 2004

Related U.S. Application Data
(60) Provisional application No. 60/496,157, filed on Aug. 19, 2003.

(51) Int. Cl. A61K 31/704

(52) U.S. Cl. 514/35; 514/39

(57) ABSTRACT

Wound healing compositions are applied intranasally to prevent or ameliorate infections and allergies. In one embodiment, the wound healing composition can be selected from the group consisting of bacitracin, polymyxin, neomycin and combinations thereof. In another embodiment, the composition can be a petroleum or natural oil based ointment with or without antibiotic components.

### REFERENCE TO RELATED APPLICATIONS

This application claims the benefit of priority of U.S. Ser. No. 60/496,157, filed Aug. 19, 2003, entitled “Use of Wound Healing Compositions For Prevention of Infections and Allergies,” the content of which are incorporated herein by reference.

### BACKGROUND OF THE INVENTION

The technical field of the invention is prevention and/or treatment of infections and allergies.

There are three known influenza-type viruses which affect human beings: Influenza A, B and C. Influenza A viruses have been isolated from many animal species in addition to humans, while the influenza B and C viruses infect mainly humans. The influenza viruses are enveloped viruses containing negative single-stranded RNA's which are segmented and encapsidated. The influenza virus envelope is characterized by the presence of two surface glycoproteins: hemagglutinin and neuraminidase. The influenza A and B virions are pleomorphic and are usually 80-120 nm in diameter. The influenza C virion has many distinctive properties and is thus distinguished from the closely related A and B virions. Infection with influenza A or B often can cause a highly contagious, acute respiratory illness.

Influenza viruses have a major impact on morbidity leading to increases in hospitalization and in visits to health care providers. High rates of hospitalization are observed for patients over 65 years of age and also for children less than 5 years of age.

There are relatively few known compounds that have significant anti-viral activity against influenza viruses. Two of these, amantadine and rimantadine, are approved in the United States for the treatment of influenza virus disease. Both compounds are most effective when used prophylactically. (See U.S. Pat. Nos. 3,152,180 and 3,352,912). However, the influenza viruses develop resistance to both compounds rapidly. In addition, both of these compounds produce strong undesired side effects.

Other methods for preventing influenza involve vaccination with an attenuated virus. However, these vaccination often result in adverse effects in the individual, particularly in the elderly or those with weak immune systems. Furthermore, the vaccine is not completely efficient in producing antibodies, and thus cannot prevent the spread of infection entirely. In addition, because the influenza virus mutates, the effect of vaccination with one form of inactivated virus does not necessarily provide long term benefits to the individual. Often repeated vaccination with different forms of the attenuated virus are required on a yearly basis.

Similar problems are also encountered in preventing bacterial infections. Again, vaccination is dependent on each individual's ability to produce antibodies in sufficient quantities to counteract systemic infections.

Accordingly, a need exists for preventing or ameliorating infections that circumvent the problems in the prior art. A need also exists for preventing or ameliorating allergies.

### SUMMARY OF THE INVENTION

The present invention is based, at least in part, on the discovery that wound healing compositions and, in particular, ointment-based, wound healing compositions, when applied intranasally, can be used to prevent or ameliorate infections such as viral infections, bacterial infections, fungal infections, as well as allergies. In particular, the invention is directed to preventing or reducing the onset of infections by reducing the probability of interaction between the infectious particle or allergen with the mucous lining of the nasal passage, and/or passage into the lungs, thereby providing a protective barrier. The method of the invention can be particularly suitable for individuals with a weak immune system who are prone to infections, and the elderly.

Accordingly, in one aspect, the invention pertains to a method for preventing or ameliorating viral infections comprising intranasal topical application of a wound healing composition to intranasal mucous membranes, such that the wound healing composition provides a barrier that prevents the viral organism from attaching to the intranasal mucous membrane, or otherwise gaining systemic entry via the intranasal passage of the subject to the lungs or sinus cavities.

In one embodiment, the viral organism is an influenza viral strain selected from the group consisting of influenza virus strain A, influenza virus strain B, and influenza virus strain C.

In one embodiment, the wound healing composition can be selected from the group consisting of bacitracin, polymyxin, neomycin and combinations thereof. In one preferred embodiment, the wound healing composition is Polysporin®, a commercially available formulation of bacitracin and polymyxin. In another preferred embodiment, the wound healing composition is Neosporin®, a commercially available formulation of neomycin, bacitracin and polymyxin.

More generally, wound healing compositions that may be useful in the present invention include a wide spectrum of antibacterial compositions: aminoglycosides such as Amikacin, Apramycin, Arbekacin, Bambermycins, Butirosin, Dibekacin, Dihdrostreptomycin, Fortimicin(s), Gentamicin, Ispamicin, Kanamycin, Micronomicin, Neomycin Undecylenate, Netilmicin, Paromomycin, Ribostamycin, Sisomicin, Spectinomycin, Streptomycin, Streptonicozid and Tobramycin; Amphenicols such as Azidamfenicol, Chloramphenicol, Chloramphenicol Palmitate, Chloramphenicol Pantothenate, Florfenicol and Thiamphenicol; Ansamycins such as Rifamide, Rifampin, Rifamycin and Rifaximin; β-Lactams, including: Carbapenems such as Imipenem; Cephalosporins such as Cefactor, Cefadroxil, Cefamandole, Cefatrizine, Cefazedone, Cefazolin, Cefixime, Cefmenoxime, Cefodizime, Cefonicid, Cefoperazone, Ceforanide, Cefotaxime, Cefotiam, Cefpimizole, Cefpirimide, Cefpodoxime Proxetil, Cefroxadine, Cefsulodin, Ceftazidime, Cefteram, Ceftezole, Ceftibuten, Ceftizoxime, Ceftriaxone, Cefuroxime, Cefuzonam, Cephacetrile Sodium, Cephalexin, Cephaloglycin, Cephaloridine, Cephalosporin, Cephalothin, Cephapirin Sodium, Cephradine and Pivcefalexin; Cephamycins such as Cefbuperazone, Cefinetazole, Cefminox, Cefetan and Cefoxitin; Monobactams such as Aztreonam, Carumonam and Tigemonam; Oxacephems such as Flomoxef and Moxolactam; Penicillins such as Amidinocillin, Amdinocillin Pivoxil, Amoxicillin, Ampicillan, Apalcillin, Aspoxicillin, Azidocillan, Azlocillan, Bacampicillin, Benzylpenicillinic Acid, Benzylpenicillin Sodium, Carbenicillin, Carfecillin Sodium, Carindacillin, Clometocill in, Cloxacill in, Cyclacillin, Dicloxacillin, Diphenicillin Sodium, Epicillin, Fenbenicillin, Floxicillin, Hetacillin, Lenampicillin, Metampicillin, Methicillin Sodium, Mezlocillin, Nafcillin Sodium, Oxacillin, Penamecillin, Penethamate Hydriodide, Penicillin G Benethamine, Penicillin G Benzathine, Penicillin G Benzhydrylamine, Penicillin G Calcium, Penicillin G Hydrabamine, Penicillin G Potassium, Penicillin G Procaine, Penicillen N, Penicillin O, Penicillin V, Penicillin V Benzathine, Penicillin V Hydrabamine, Penimepicycline, Phenethicillin Potassium, Piperacillin, Pivapicillin, Propicillin, Quinacillin, Sulbenicillin, Talampicillin, Temocillin and Ticarcillin; Lincosamides such as Clindamycin and Lincomycin; Macrolides such as Azithroimycin, Carbomycin, Clarithromycin, Erythromycin, Erythromycin Acistrate, Erythromycin Estolate, Erythromycin Glucoheptonate, Erythromycin Lactobionate, Erythromycin Propionate, Erythromycin Stearate, Josamycin, Leucomycins, Midecamycins, Miokamycin, Oleandomycin, Primycin, Rokitamycin, Rosaramicin, Roxithromycin, Spiramycin and Troleandomycin; Polypeptides such as Amphomycin, Bacitracin, Capreomycin, Colistin, Enduracidin, Enviomycin, Fusafungine, Gramicidin(s), Gramicidin S, Mikamycin, Polymyxin, Polymyxin B-Methanesulfonic Acid, Pristinamycin, Ristocetin, Teicoplanin, Thiostrepton, Tuberactinomycin, Tyrocidine, Tyrothricin, Vancomycin, Viomycin, Viomycin Pantothenate, Virginiamycin and Zinc Bacitracin; Tetracyclines such as Apicycline, Chlortetracycline, Clomocycline, Demeclocycline, Doxycycline, Guamecycline, Lymecycline, Meclocycline, Methacycline, Minocycline, Oxytetracycline, Penimepicycline, Pipacycline, Rolitetracycline, Sancycline, Senociclin and Tetracycline; and other antibiotics such as Cycloserine, Mupirocin and Tuberin.

Other antibacterial, wound healing compositions that may be useful in the present invention further include 2,4-Diaminopyrimidines such as Brodimoprim, Tetroxoprim and Trimethoprim; Nitrofurans such as Furaltadone, Furazolium Chloride, Nifuradene, Nifuratel, Nifurfoline, Nifurpirinol, Nifurprazine, Nifurtoinol and Nitrofurantoin; Quinolones and Analogs such as Amifloxacin, Cinoxacin, Ciprofloxacin, Difloxacin, Enoxacin, Fleroxacin, Flumequine, Lomefloxacin, Miloxacin, Nalidixic Acid, Norfloxacin, Ofloxacin, Oxolinic Acid, Pefloxacin, Pipemidic Acid, Piromidic Acid, Rosoxacin, Temafloxacin and Tosufloxacin; Sulfonamides such as Acetyl Sulfamethoxypyrazine, Acetyl Sulfisoxazole, Azosulfamide, Benzylsulfamide, Chloramine-B, Chloramine-T, Dichloramine T, Formosulfathiazole, N² Formylsulfisomidine, N²-β-D-Glucosylsulfanilamide, Mafenide, 4′-(Methylsulfamoyl)sulfanilanilide, p-Nitrosulfathiazole, Noprylsulfamide, Phthalylsulfacetamide, Phthalylsulfathiazole, Salazosulfadimidine, Succinylsulfathiazole, Sulfabenzamide, Sulfacetamide, Sulfachlorpyridazine, Sulfachrysoidine, Sulfacytine, Sulfadiazine, Sulfadicramide, Sulfadimethoxine, Sulfadoxine, Sulfaethidole, Sulfaguanidine, Sulfaguanol, Sulfalene, Sulfaloxic Acid, Sulfamerazine, Sulfameter, Sulfamethazine, Sulfamethizole, Sulfamethomidine, Sulfamethoxazole, Sulfamethoxypyridazine, Sulfametrole, Sulfamidochrysoidine, Sulfamoxole, Sulfanilamidomethanesulfonic Acid Triethanolamines, Sulfanilamide, 4-Sulfanilamidosalicylic Acid, N-Sulfanilylsulfanilamide, Sulfanilylurea, N-Sulfanilyl-3,4-xylamide, Sulfanitran, Sulfaperine, Sulfaphenazole. Sulfaproxyline, Sulfapyrazine, Sulfapyridine, Sulfasomizole, Sulfasymazine, Sulfathiazole, Sulfathiourea, Sulfatolamide, Sulfisomidine and Sulfisoxazole; Sulfones such as Acedapsone, Acediasulfone, Acetosulfone Sodium, Dapsone, Diathymosulfone, Glucosulfone Sodium, Solasulfone, Succisulfone, Sulfanilic Acid, p-Sulfanilylbenzylamine, p,p′-Sulfonyldianiline-N.N′digalactoside, Sulfoxone Sodium and Thiazolsulfone; and others such as Clofoctol, Hexedine, Methenamine, Methenamine Anhydromethylene-citrate, Methenamine Hippurate, Methenamine Mandelate, Methenamine Sulfosalicylate, Nitroxoline and Xibomol. When the wound healing composition contains one or more antibiotics, e.g., bacitacin, polymixin or neomycin, it can be more effective in preventing bacterial infections because it not only provides a physical barrier, but also kills the bacteria on contact.

The wound healing compositions of the present invention can also be formulated without active antibiotics. For certain preventive applications, an ointment or salve alone will suffice to prevent or inhibit infections and/or allergenic reactions. Such ointment based wound healing compositions can include natural oils, petroleum jelly (petrolatum), stearates, alcohols, glycols, glycerines, waxes, gels, acrylates, and plant extracts.

Examples of natural oils and plant extracts further include aloe extracts, almond oil, avocado oil, carrot oil, sunflower oil, safflower oil, coconut oil, evening primrose oil, olive oil, cocoa butter, pecan oil, tea tree oil, rose hips oil, canola oil, green tea extract, honey extract, lemon extract, fennel extract, sage extract, yarrow extract, jojoba extract, chamomile extract, echinacea extract, lavender extract, sesame oil, grapeseed oil, cucumber extract, rosemary extract, horse chestnut oils, dandelion extract, coltsfoot extract, sea kelp extract, and bee pollen:

The ointments of the present invention can further include palm stearates, cetearyl alcohol, polysorbate 60, stearic acid, cetyl alcohol, isopropyl palmitate, propylene glycol methyl paraben, propyl paraben, hydantoins, triethanolamine, vitamin E, imidazolidinyl urea, panthenol (pro-vitamin B5), lecithin, silica, tocopheryl acetate, presevatives, water and fragrances.

The wound healing composition can be applied topically, and preferably intranasally. The number of times the wound healing composition is applied can vary as necessary based on the need of the subject. Thus, the wound healing composition can be applied intranasally at least once a day, at least two times a day, at least three times a day, or as many times as is necessary to prevent or ameliorate the infection, such as a viral infection, a bacterial infection, or a fungal infection.

In another aspect, the invention pertains to a method for preventing or ameliorating allergies caused by allergens comprising intranasal topical application of a wound healing composition to intranasal mucous membranes, such that the wound healing composition provides a barrier that prevents the allergen from attaching to the intranasal mucous membrane, or otherwise gaining systemic entry via the intranasal passage of the subject to the lungs or sinuses.

The allergens can be selected from the group consisting of pollens, dust, dust mites, mold spores, and animal hair. The wound healing composition can be selected from the group consisting of bacitracin, polymixin, neomycin and combinations thereof. In a preferred embodiment, the wound healing composition is Polysporin®. In another preferred embodiment, the wound healing composition is Neosporin®. In yet another preferred embodiment, the composition is an ointment without antibiotic drugs. Again, the number of times the wound healing composition is applied can vary as necessary based on the need of the subject. Thus, the wound healing composition can be applied intranasally at least once a day, at least two times a day, at least three times a day, or as many times as is necessary to prevent or ameliorate the allergy.

### DETAILED DESCRIPTION

So that the invention is more clearly understood, the following terms are defined:

The term “infection” is an art recognized term for a contamination, or the establishment of a microorganism in a host after invasion of the host.

The term “subject” as used herein refers to any living organism in which an immune response is elicited. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

The term “allergy” is also an art recognized term referring to an exaggerated or pathological reaction to substances, or physical states. In particular, the term “allergy” refers to the abnormally high sensitivity to certain substances, such as pollens, or microorganisms. Common indications of allergy may include sneezing, itching, watery eyes, and respiratory problems.

The term “allergen” as used herein refers any substance that induces an allergy, or an allergic reaction. Examples of allergens include, but are not limited to, dust, dust mites, and pollens.

The term “wound healing composition” as used herein refers to a formulation designed to treat wounds, lesions, or injuries caused by physical or chemical means. The wound healing process comprises a series of steps where the injured tissue is repaired or regenerated. This usually involves three steps (a) an inflammation stage (0-3 days), (b) a cellular proliferation phase (3-12 days), and (c) a remodeling phase (3-6 months). Typically, the wound healing composition has at least one active ingredient that helps to repair the wound. Examples of active ingredients include, but are not limited to, bacitracin, neomycin, and polymixin (e.g., polymixin B sulfate). The wound healing composition may also contain inactive ingredients that are typically required for formulation (See e.g., U.S. Pat. No. 5,652,274, incorporated herein by reference). In one embodiment, the wound healing compositions is Neosporin®. In another embodiment, the wound healing composition is Polysporin®.

The term “nasal passage” or “intranasal cavity” are used interchangeably herein and refer to the walls of the inside of the nose that are coated with respiratory mucous membranes which contain innumerable tiny hair-like cells that act to move waves of mucus toward the throat. Dust, other allergens, bacteria, viruses and other particles inhaled from the air are trapped by the mucus in the nose, carried back, swallowed and dropped into the gastric juices or alternatively carried into the lungs or sinus cavities. The wound healing composition can be applied to the nasal passage to prevent or ameliorate infections or allergies in a subject by providing a barrier to intranasal passage or simply by trapping the infectious particle or allergen or by having it stick to, or adhere to, an ointment component of the composition.

The term “intranasal mucous membrane” as used herein refers a tissue lining the inside of the nasal passage that is rich with mucus glands, or which secretes mucus.

The term “ameliorates” as used herein refers to a change in state from a first to a second state, where the first state is the condition of the nasal passage subject prior to application of the wound healing composition, and the second stage is the condition of the subject after application of the wound healing composition, where the condition in the second stage is better or improved as a result of applying the wound healing composition.

The invention is described in more detail in the following subsections:

I. Infections and Allergies

The invention pertains to using wound healing compositions for the amelioration or prevention of infections and allergies. Any number of infections that arise through infectious particles entering into the nasal passage and subsequently interacting with the nasal mucosal lining, lungs or sinuses can be prevented using the methods of the invention. The infectious particles can be viral particles, bacterial particles, allergens, fungal particles, and the like. In one embodiment, the infection is caused by a viral particle (e.g., a common cold or an influenza virus), and is prevented or ameliorated by the wound healing composition applied to the intranasal cavity.

Influenza is extremely prevalent and highly infectious. Most people are infected by airborne germs. The influenza virus belongs to orthomyxoviridae family, and is classified into three types, A, B, and C, on the basis of the differences in serotypes of the nucleoprotein (NP) and the membrane protein M2. Amino acid sequences of the RNA polymerase are conserved in the influenza A, B, C or a mutant thereof.

Of the influenza viruses, the influenza A virus undergoes a substantial change in antigenecity, and prevails above all others. In view of an acute infectivity, the influenza A virus is most malignant. As an antiviral agent for the influenza A virus, amantadine or rimantadine are known, but these cannot cope with mutants and have strong side effects.

Treatment with an inactivated vaccine has been attempted, but there is a difference between the inactivated vaccine and an antibody generated by a natural infection, thus some of the benefits of vaccination are not always apparent in a population of individuals. Furthermore, the vaccine is not completely efficient in producing antibodies, and thus cannot prevent the spread of infection entirely. These vaccines may cause an adverse reaction in some individuals, particularly those with a weak immune system or the elderly. Thus, the methods of the invention can be used to treat or prevent influenza-like viral infections by applying the wound healing composition to the intranasal cavity.

The common cold is likewise prevalent and infectious. The many strains of cold viruses have largely eluded preventive vaccine measures. However, the methods of the invention can also be used to prevent cold virus infections by applying the wound healing composition to the intranasal cavity.

The skilled artisan will appreciate that the methods of the invention can also be used to ameliorate or prevent various other viral infections, bacterial infections, fungal infections, and the like that occur through the nasal passage.

In another aspect, the invention pertains to ameliorating or preventing allergies that arise through the nasal passage. There are several types of allergens that cause allergies in individuals. For example, allergens such as dust, dust mite, pollen, animal hair, and the like. House dust is the source of troublesome symptoms of allergy for a number of people who may be constantly bothered by sneezing and a runny nose or by wheezing and shortness of breath.

Hayfever is a well known disease which affects some 5% of the human population. The characteristic symptoms are recurrent coryzae and sneezing, excessive nasal secretion, swollen eyelids, light-sensitivity, all of these often in combination with widespread urticarial eruptions, bronchial spasm, asthma, and a general feeling of malaise and physical exhaustion. These individuals are allergic to the pollen of flowering grasses, trees or other plants, and that the disease symptoms become apparent under climatological conditions favoring a high pollen count of the air.

The methods of the invention can also be used to treat or prevent allergies by intranasal application of the wound healing composition to the nasal passage. The wound healing composition prevents, or protects against the interaction of the allergen with the muscosal lining of the nasal passage and further trap the allergen from passing into the lungs or sinuses. The ointments of the present invention are particularly useful in trapping infectious particles or allergens and preventing their passage into more vulnerable areas, such as the lungs and sinus cavities.

II Wound Healing Compositions

This invention pertains to using wound healing compositions to prevent or ameliorate infections (e.g., viral and bacterial infections) that arise through the nasal passage, as well as preventing or ameliorating allergies that arise due to contact of an allergen (e.g., dust, pollen) with, or passage through, the nasal passage. The wound healing composition may act to line the nasal passage such that the infectious particles or allergens are prevented from interacting with the nasal passage mucosal membrane and greatly reduces their passage into the lungs and/or the sinus cavities. Thus, the wound healing composition may act by providing a barrier between the nasal passage mucosal membrane lining and the particles or allergens, or otherwise protect the lungs and sinuses from such particles, that are inhaled into the nasal passage. The wound healing composition may also act by maintaining the nasal passage in a moist condition, thereby preventing the appearance of abrasions or cuts into which the particles or allergens may attach and enter. Thus, the wound healing composition prevents the onset of an infection or allergy.

Wound healing compositions are described in U.S. Pat. No. 5,652,274 (incorporated herein by reference). In one preferred embodiment, the wound healing composition comprises at least one composition selected from the following active ingredients: bacitracin, neomycin, and polymixin (e.g., polymixin B sulfate). In one preferred embodiment, the combination of active ingredients in the wound healing composition can be bacitracin, neomycin, and polymixin B sulfate. In another preferred embodiment, the combination of active ingredients in the wound healing composition can be bacitracin and polymixin B sulfate. When the wound healing composition contains one or more antibiotics, e.g., bacitacin, polymixin or neomycin, it can be more effective in preventing bacterial infections because it not only provides a physical barrier, but also kills the bacteria on contact. In another preferred embodiment, the composition is a petroleum based ointment, with or without, antibiotic components.

III. Application of the Wound Healing Compositions

In addition to the active ingredients (e.g., bacitracin, neomycin, and polymixin B sulfate) in the wound healing composition, the wound healing composition typically has incorporated into it, a pharmaceutically acceptable carrier. As used herein, “pharmaceutically acceptable carrier” includes any and all solvents, dispersion media, coatings, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the wound healing composition.

In particular, the wound healing composition can further comprise a pyruvate selected from the group consisting of pyruvic acid, pharmaceutically acceptable salts of pyruvic acid, and mixtures thereof; an antioxidant; and a mixture of saturated and unsaturated fatty acids wherein the fatty acids are those fatty acids required for the repair of cellular membranes and resuscitation of mammalian cells.

The wound healing compositions may be manufactured in a variety of forms. These include, for example, liquid, semi-solid and solid dosage forms, such as liquid solutions (e.g., injectable and infusible solutions), dispersions or suspensions. The preferred form depends on the intended mode of administration and therapeutic application. In a preferred embodiment, the wound healing composition is used for topical application, such as a petroleum-based ointment. Such topical formulations can further include a base of cocoa butter, cottonseed oil, olive oil, tocopheryl acctide and/or petrolatum. In another embodiment, the wound healing composition is in the form of a nasal spray.

A “therapeutically effective amount” of the wound healing composition refers to an amount effective, at dosages and for periods of time necessary, to achieve the desired therapeutic result (i.e., the prevention of amelioration of an infection or allergy). A therapeutically effective amount of the of the wound healing composition may vary according to factors such as the age, sex, and weight of the individual. A therapeutically effective amount is also one in which any toxic or detrimental effects of the wound healing composition are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount. In a preferred embodiment, the wound healing composition is applied intranasally between 1 to about 6 times a day, more preferably about 1 to about 4 time a day, even more preferably about 1 to about 3 times a day, 1 to about 2 times a day, and most preferably once a day. If the wound healing composition is in the form of an ointment, cream or salve, the composition can be applied by using an application device such as a Q-tip or similar cotton swab, to gently apply the wound healing composition broadly into the nasal passage.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

### EXAMPLES

### Example 1

Prevention of Viral Infection with Wound Healing Compositions

This example describes the effect on at least two human individuals who have routinely applied a wound healing composition to the intranasal cavity. In particular, the present example describes the use of Polysporin as a wound healing composition.

Subject 1:

Subject 1 is a 65 year old male who has a weak immune system and a high propensity for contracting influenza and colds. Subject 1 routinely suffered from about 5-6 colds and infections a year, and has had typhoid fever, paratyphoid fever, and scarlet fever. In addition, the spleen in subject 1 had been removed, thereby further weakening the immune system.

For three years, subject 1 has been applying Polysporin on a daily basis to the intranasal cavity using a Q-tip cotton swab. The Polysporin is applied once a day throughout the year. After about 6 months, Subject 1 experienced no colds or infections. Subject 1 continued this routine for 3½ years, and has experienced only one cold during this time.

Subject 2:

Subject 2 is a 79 year old male who contracted influenza and colds and infections about 2-3 times a year. Subject 2 also dives, and often contracted colds and infections after diving. For about 18 months, Subject 2 has been applying Polysporin on a daily basis to the intranasal cavity using a Q-tip cotton swab. The Polysporin was applied once a day. After about 18 months, Subject 2 has experienced no colds and infections.

These results demonstrate that wound healing compositions applied to the intranasal cavity prevent or ameliorate infection by infectious particles.

### Example 2

Clinical Trials for Prevention of Viral Infection with Wound Healing Compositions

This example shows that the methods of the present invention can be used as a preventive technique designed to substantially reduce the onset of viral and bacterial infections, as well as allergies.

An immune-compromised group, who normally contracts more colds and flus from viruses and bacteria than the average public, can be used as test subjects. Nursing homes provided the ideal group of subjects. A small amount of Neosporin can be applied to the nasal cavities of each patient once a day in the morning. A Q-tip can be used to apply the substance with the whole process taking seconds and one that can be administered to the patients as part of the routine morning attention that each patient receives.

As most infections enter through the nasal cavity, the application of this substance, with its antibiotic properties kills the bacteria before the bacteria enters the system. Likewise, the ointment property causes enough of the viral load to stick to it so that an insufficient amount enters the system to cause a viral infection. The ointment may also cut down on the amount of allergens that enter the system, thus also retarding allergic type reactions.

In one approach, the trial group can be split into three subgroups. The first group would receive the daily (morning) application of Neosporin. The second group would receive the application of Vaseline. The third group would receive a placebo (i.e., colored water). Records of the participants' pre-trial history of the number of infections for a year or two would be prepared and then compared with the results listing the number of infections contracted during the trial period. The reduction in the number of infections during the trial period for the participants medicated with either Neosporin or Vaseline as compared with their recent pre-trial history and/or the placebo group, will provide proof of the efficacy.

The method of the invention provides an effective way to reduce infection, reduce medical costs, and reduce the suffering caused by colds, flus, sinus infections, allergies and their complications such as pneumonia and bronchitis.

1. A method for ameliorating viral infections caused by viral organisms comprising intranasal topical application of a wound healing composition to intranasal mucous membranes, such that the wound healing composition provides a barrier that prevents the viral organism from systemic entry via the intranasal passage of the subject.

2. The method of claim 1, wherein the viral organism is an selected from the group consisting of influenza virus strain A, the influenza virus strain B, and the influenza virus strain C.

3. The method of claim 1, wherein the wound healing composition comprises at least one composition selected from the group consisting of bacitracin, polymixin, and neomycin.

4. The method of claim 1, wherein the wound healing composition comprises Polysporin®.

5. The method of claim 1, wherein the wound healing composition comprises Neosporin®.

6. The method of claim 1, wherein the wound healing composition comprises a petroleum-based ointment.

7. The method of claim 1, wherein the wound healing composition comprises petroleum-jelly.

8. The method of claim 1, wherein the wound healing composition is applied intranasally at least once a day.

9. The method of claim 1, wherein the wound healing composition is applied intranasally at least two times a day.

10. The method of claim 1, wherein the wound healing composition is applied intranasally at least three times a day.

11. A method for preventing or ameliorating allergies caused by allergens comprising intranasal topical application of a wound healing composition to intranasal mucous membranes, such that the wound healing composition provides a barrier that prevents the allergen from systemic entry via the intranasal passage of the subject.

12. The method of claim 11, wherein the allergen is an selected from the group consisting of pollens, dust, dust mites, mold spores, and animal hair.

13. The method of claim 11, wherein the wound healing composition comprises at least one composition selected from the group consisting of bacitracin, polymixin, and neomycin.

14. The method of claim 11, wherein the wound healing composition comprises Polysporin®.

15. The method of claim 11, wherein the wound healing composition comprises Neosporin®.

16. The method of claim 11, wherein the wound healing composition comprises a petroleum-based ointment.

17. The method of claim 11, wherein the wound healing composition comprises petroleum-jelly.

18. The method of claim 11, wherein the wound healing composition is applied intranasally at least once a day.

19. The method of claim 11, wherein the wound healing composition is applied intranasally at least two times a day.

20. The method of claim 9, wherein the wound healing composition is applied intranasally at least three times a day.

21. A method for preventing bacterial infections caused by bacterial organisms comprising intranasal topical application of a wound healing composition to intranasal mucous membranes, such that the wound healing composition provides a barrier that prevents the bacterial organism from systemic entry via the intranasal passage of the subject.

22. The method of claim 21, wherein the bacterial organism a streptococcus bacterium.

23. The method of claim 21, wherein the wound healing composition comprises at least one composition selected from the group consisting of bacitracin, polymixin, and neomycin.

24. The method of claim 21, wherein the wound healing composition comprises Polysporin®.

25. The method of claim 21, wherein the wound healing composition comprises Neosporin®.

26. The method of claim 21, wherein the wound healing composition comprises a petroleum-based ointment.

27. The method of claim 21, wherein the wound healing composition comprises petroleum-jelly.

28. The method of claim 21, wherein the wound healing composition is applied intranasally at least once a day.

29. The method of claim 21, wherein the wound healing composition is applied intranasally at least two times a day.

30. The method of claim 21, wherein the wound healing composition is applied intranasally at least three times a day.
